(19) United States
(12) Patent Application Publication
(10) Pub. No.: US 2005/0123635 A1
(43) Pub. Date: Jun. 9, 2005

(54) Cannabis aerosol

(76) Inventors: John Jackson McAughey, Didcot (GB); Ian Anthony Marshall, Chipping Norton (GB)

Correspondence Address: LAW OFFICES OF WILLIAM H. HOLT, 12311 HARBOR DRIVE, WOODBRIDGE, VA, 22192, US

(21) Appl. No.: 10/725,439

(22) Filed: Dec. 3, 2003

(51) Int. Cl. A61K 35/78; A24F 3/00

(52) U.S. Cl. 424/774; 131/185

(57) ABSTRACT

Cannabis is a narcotic substance derived from the hemp plant (Cannabis indica or Cannabis sativa), and a range of derivatives and extracts are known. An aerosol of cannabis can be made by vaporising cannabis at an elevated temperature, causing the cannabis vapour to flow to a region at lower temperature at which the vapour would be supersaturated, generating seed nuclei of particle size less than 0.5 μm, and mixing the seed nuclei with the vapour. Heterogeneous nucleation occurs due to the seed nuclei, so that droplets of a desired diameter and concentration can be obtained. The seed nuclei may for example be derived by passing air over molten sodium chloride.

The invention relates to an apparatus and to a method for providing cannabis, cannabis oils, and cannabis derivatives in the form of an aerosol suitable for inhalation.

There is currently an increasing interest in the potential use of cannabis and its derivatives for therapeutic use. However cannabis oils are of high viscosity, so that existing nebulizer mechanisms cannot provide a high concentration of aerosol droplets. Inhalation of vapour, on the other hand, is an inefficient way of providing a therapeutic dose to the lungs because much of the vapour will condense out on other surfaces in the air passage ways or will be exhaled, while if the vapour is generated by smoking, toxic smoke components will also be generated by pyrolysis. With other pharmaceuticals it is known that provision of fine droplets, say less than 5 μm in diameter, is an efficient route to ensure delivery to the lung, so that it would be desirable to be able to generate an aerosol of particles or droplets of cannabis of such a size at a high concentration.

Cannabis is a narcotic substance derived from the hemp plant (Cannabis indica or Cannabis sativa); the leaves and stalks of these plants may be referred to as hashish or marijuana. The term cannabis may refer to the female flowering heads of hemp, or to a resin obtained from the flowering heads that may also be referred to as cannabin. Derived materials include cannaboid, an alkaloid cannabine, and an oil cannabinol. In this specification these, and other derivatives, are referred to generically as cannabis.

According to the present invention there is provided a method of forming an aerosol of cannabis, the method comprising vaporising cannabis at an elevated temperature, causing the cannabis vapour to flow with a carrier gas to a region at a lower temperature at which the vapour would be supersaturated, generating seed nuclei of particle size less than 0.5 μm, and mixing the seed nuclei with the cannabis vapour and the carrier gas.

The provision of seed nuclei enables heterogeneous nucleation to occur, so that droplets of a desired diameter and concentration can be obtained. The seed nuclei may be mixed with the cannabis vapour before it flows to the lower temperature region.

The seed nuclei may for example be generated by passing air over a bath of molten sodium chloride, or molten silver, or by using an electrically heated wire for example of palladium.

The invention also provides an apparatus for performing this method. Preferably the cannabis is introduced into the region at elevated temperature at a controlled rate, for example using a syringe. When using cannabis oil, the boiling point of the oil is about 280° C., but the elevated temperature may be between 180° C. and 280° C., preferably between 200° C. and 260° C., for example 250° C., at which temperature the oil will evaporate. The apparatus may also include additional vaporising means for vaporising other components, such as glycerol, which are also mixed with the carrier gas so that the resulting aerosol droplets contain both cannabis and the other components.

By adjusting the flow rates, aerosol droplets of a desired size may be obtained. For example substantially monodisperse droplets have been obtained at diameters in the range 0.7 μm to 2.0 μm. The concentration of the aerosol may be up to 100 mg/litre, for example 50 mg/litre.

The invention will now be further and more particularly described, by way of example only, and with reference to the accompanying drawings in which:

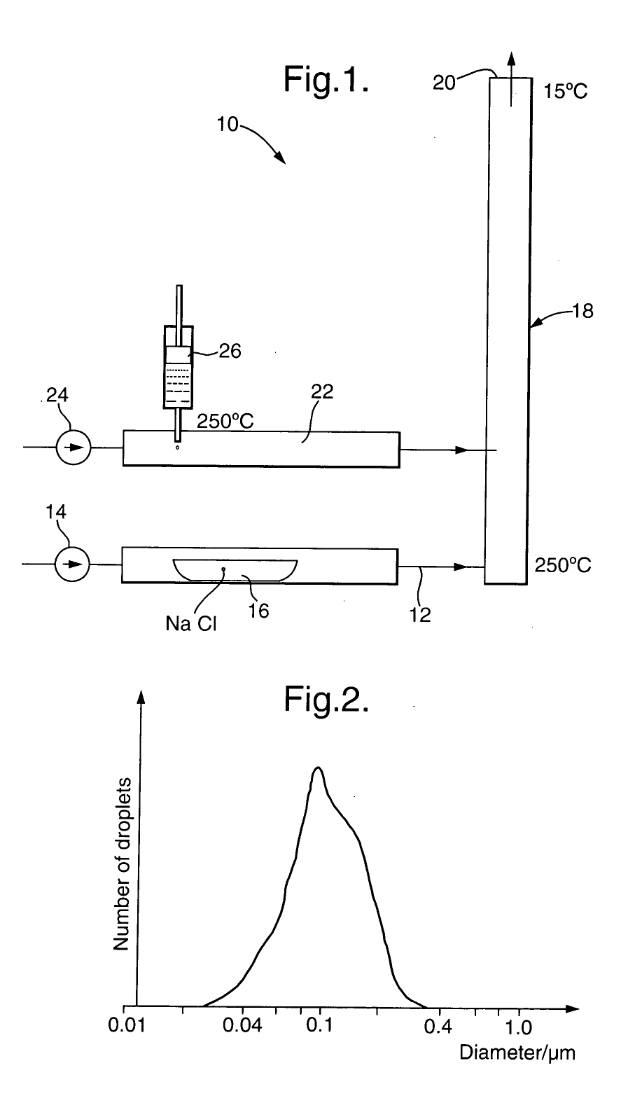

FIG. 1 shows a diagrammatic view of an apparatus for generating a cannabis aerosol; and

FIG. 2 shows a particle size distribution for particles made with an apparatus as in FIG. 1.

Referring to FIG. 1, an apparatus 10 for generating an aerosol comprises a first gas flow line 12 along which air is caused to flow by a pump 14, and within which is a bath 16 containing sodium chloride and heated electrically to a temperature of 850° C. at which the sodium chloride is molten. Evaporation of sodium chloride creates very small particles (typically no larger than 50 nm) of sodium chloride in the air stream emerging from the first gas flow line 12. This air stream then flows along a duct 18 whose walls are held at a controlled temperature that decreases along its length from about 250° C. at the junction with the flow line 12, to 15° C. at its open end 20.

A second gas flow line 22 also communicates with the duct 18 near the high temperature end, and air is caused to flow along this line 22 by a pump 24. The flow line 22 is held at a temperature of 250° C. A syringe 26 is arranged to introduce cannabis oil at a steady rate into the line 22, in which it evaporates.

The two air flows mix within the duct 18, the resulting mixture initially containing cannabis vapour and particles of sodium chloride. As the air cools the cannabis condenses onto the sodium chloride particles to form an aerosol in which the droplets are of substantially uniform size, and consist primarily of cannabis. The droplet size is determined by the particle concentration, and by the vapour concentration, and can therefore be controlled by controlling the air flow through the two lines 12 and 22, the feed rate of the cannabis through the syringe 26, and the temperature of the sodium chloride bath 16.

As an illustration, droplets have been made in this manner (using glycerol in place of cannabis) at sizes between 0.7 and 2.04 μm, the particles being substantially monodisperse in each case, and at concentrations up to 50 mg/litre; a particle size distribution for such aerosol droplets is shown in FIG. 2.

It will be appreciated that the cannabis might instead be supplied in the form of a solution in a suitable solvent, so that both the solvent and the cannabis evaporate when they are introduced into the line 22, and as a consequence the resulting aerosol droplets emerging from the outlet 20 will be of both cannabis and the solvent. Alternatively the apparatus might include a third gas flow line (not shown) similar to the second gas flow line 22, whereby a vapour of another liquid (such as glycerol) may be introduced into the duct 18, so the resulting aerosol droplets emerging from the outlet 20 will contain both cannabis and this other liquid. Provision of another liquid in one or other of these ways may enable particles of a desired larger size to be generated.

The wall of the duct 18 may be cooled actively, using one or more heat exchange jackets through which coolant fluids are passed, or may be merely exposed to the surrounding air so the wall loses heat by natural convection.

1. A method of forming an aerosol of cannabis, the method comprising vaporising cannabis at an elevated temperature, causing the cannabis vapour to flow with a carrier gas to a region at a lower temperature at which the vapour would be supersaturated, generating seed nuclei of particle size less than 0.5 μm, and mixing the seed nuclei with the cannabis vapour and the carrier gas.

2. A method as claimed in claim 1 wherein the seed nuclei are generated by passing a gas over a bath of molten material.

3. A method as claimed in claim 2 wherein the molten material is sodium chloride.

4. A method as claimed in claim 1 wherein the seed nuclei are generated by passing a gas over an electrically heated wire.

5. An apparatus for forming an aerosol of cannabis, the apparatus comprising means for vaporising cannabis at an elevated temperature, means for causing the cannabis vapour to flow with a carrier gas to a region at lower temperature at which it becomes supersaturated, means to generate seed nuclei of particle size less than 0.5 μm, and means to mix the seed nuclei with the cannabis vapour and the carrier gas.

6. An apparatus as claimed in claim 5 wherein the cannabis is introduced into the vaporising means at a controlled rate.

7. An apparatus as claimed in claim 5 also comprising additional vaporising means for vaporising another liquid whose vapour also mixed with the cannabis vapour so that the resulting aerosol droplets contain both cannabis and the other liquid.

8. An apparatus as claimed in claim 6 also comprising additional vaporising means for vaporising another liquid whose vapour also mixed with the cannabis vapour so that the resulting aerosol droplets contain both cannabis and the other liquid.
